(19) United States
(12) Patent Application Publication
(10) Pub. No.: US 2023/0183633 A1
(43) Pub. Date: Jun. 15, 2023

(54) OFFSHORE MOBILE PLATFORM FOR ELECTROCHEMICAL OCEAN IRON FERTILIZATION AND HYDROGEN GAS GENERATION

(71) Applicants: Northeastern University, Boston, MA (US); Woods Hole Oceanographic Institution, Woods Hole, MA (US)

(72) Inventors: Amir Taqieddin, Malden, MA (US); Akram Alshawabkeh, Franklin, MA (US); Kenneth O. Buesseler, Falmouth, MA (US)

(21) Appl. No.: 17/994,664

(22) Filed: Nov. 28, 2022

Related U.S. Application Data
(60) Provisional application No. 63/288,771, filed on Dec. 13, 2021.

(51) Int. Cl. C12M 1/42; B63B 35/00; C25B 1/04

(52) U.S. Cl. CPC C12M 35/08; B63B 35/00; C25B 1/04; B63B 2035/006

(57) ABSTRACT

An ocean iron fertilization (OIF) method and system for electrochemically controlled release of iron in an ocean to stimulate growth of phytoplankton to increase CO₂ sequestration by the ocean. The system includes a cathode submerged or floating in the ocean; an iron or iron-producing anode submerged or floating in the ocean spaced apart from the cathode; and a power supply unit connected to the cathode and the anode. The power supply unit drives electric current between the cathode and the anode such the anode generates oxygen (O₂) and ferrous iron through electrolysis to be released in the ocean, and the cathode produces hydrogen (H₂) and hydroxide (OH—) species through an electrochemical reaction at the cathode.

### CROSS REFERENCE TO RELATED APPLICATION

This application claims priority from U.S. Provisional Patent Application No. 63/288,771 filed on Dec. 13, 2021 entitled Offshore Mobile Platform for Electrochemical Ocean Iron Fertilization & Hydrogen Gas Generation, which is hereby incorporated by reference.

### BACKGROUND

The ocean is a large sink of atmospheric carbon dioxide (CO₂) since it can uptake 25% of the emitted CO₂ by human activities (i.e., about 2 petagrams of carbon per year)¹. Ocean iron fertilization (OIF) is one of several proposed methods to sequestrate atmospheric CO₂ and mitigate its effect on the global climate². Releasing iron at the upper level of the ocean (e.g., within the first 100 meters from the surface) stimulates the growth rate of phytoplankton, which are bacteria, protists, and plants in some areas of the ocean. Phytoplankton consume the atmospheric CO₂ and release oxygen (O₂) using photosynthesis. The consumed CO₂ is proportional to the growth rate of phytoplankton, which depends on the available nutrients in the ocean such as iron. Experimental measurements have shown that increasing the iron concentration by 2 nM could almost double the specific growth rate of phytoplankton³. The expansion in the ecosystem of phytoplankton is proportional to the CO₂ uptake in the ocean^{2,4,5} since the photosynthesis process is linearly proportional to the phytoplankton's cell volume5. This can have a major positive impact on global warming and climate change by stimulating the carbon (C) sequestration by expanding the growth and uptake of CO₂ by phytoplankton and assuming that some fraction of the carbon reaches the deep ocean where it is stored for long periods of time out of contact with the atmosphere. For this purpose, earlier studies proposed different approaches to enhance the iron availability in the ocean for phytoplankton. Lauderdale et al.—2020 hypothesized that binding the ocean iron with organic molecules enhances the iron bioavailability⁶. The authors claim that this approach could form a reinforcing cycle between the biological activity of phytoplankton and iron cycling. However, this approach focuses only on the iron cycling in the ocean rather than increasing the ocean iron concentration through fertilization. Alternatively, Emerson-2019 proposed using biogenic iron dust for OIF⁷. The dust is made from Fe-oxides, which are produced by chemosynthetic iron-oxidizing bacteria. Then the formed dust would be dispersed, at altitude by an aircraft, into the open ocean. The author highlights that an extensive campaign of laboratory testing is needed to investigate the reactivity of the proposed biogenic oxide in both the atmosphere and the ocean. Therefore, in addition to the complexity of this approach, there could be restrictive legalization of dispersing dust in the atmosphere due to its unknown effect on atmospheric chemistry. To sum up, there is no well-established technique that has been optimized for the process of using OIF for C sequestration. Therefore, it would be desirable to find an efficient technique for OIF. Various embodiments disclosed herein relate to techniques combining both OIF and hydrogen (H₂) gas generation in seawater using electrochemical reactions.

### BRIEF SUMMARY OF THE DISCLOSURE

In accordance with one or more embodiments, a novel offshore mobile platform is disclosed for OIF and H₂ gas generation using electrochemical processes. The platform uses an iron or iron-producing anode to generate ferrous iron for phytoplankton via electrolysis. In addition, the electrochemical reaction at the cathode produces H₂ and hydroxide (OH—) species. The generated H₂ gas at the cathode side is collected and stored on the platform for future reuse as a fuel (e.g., an energy source for the platform or transfer to onshore). The produced OH-increases the alkalinity of the ocean. Alternatively, the generated OH— can be converted to oxygen (O₂) gas and coupled with any possible oxygen evolution at the anode as part of the iron electrolysis. The platform and its reactions can be powered by a variety of energy sources, including traditional electric sources (e.g., mechanical power generator or charged batteries onboard the platform or ship), renewable energy sources (e.g., solar, tidal, blue energies), or by reusing the generated and stored H₂ as a fuel.

The design of the mobile platform provides extensive control over handling, transport, and running the platform. The platform can be either self-operated and free-standing in the ocean (i.e., floating and driven by the ocean current), or tugged by an external device such as a ship, underwater remote operating vehicle (ROV), or any floating device. Alternatively, the platform can be installed onboard a ship and carried out across the ocean to distribute the generated iron over a large scale. In these different scenarios, the electrodes can be either submerged or floating. Further, the electrode configuration and stacking can have various designs to provide more flexibility in handling the electrochemical reactions. The variety in selecting the shape and configuration of the electrode offer high flexibility, including applying the electrochemical process in a one-, two-, or three-dimensional (i.e., 1D, 2D, or 3D) arrangement, and handling the electrode offshore. Moreover, the electrochemical processes within the platform can be temporally varying to ensure efficient OIF and avoid any saturation in the growth rate of phytoplankton. This allows more accessibility to control the reaction and supply rates. For example, the OIF process can be applied continuously, intermittently (e.g., applied, stopped, or reversed in polarity over time), or as a function of time (e.g., simple pulse/alternating function). The applied current can be controlled by using a power supply unit on the platform. This electrochemical platform offers a wide range of flexible and dynamic features that allow the implementation and engineering of different processes. For example, the platform functionality is not restricted to OIF and H₂ production but can be used in other electrochemical applications such as seawater desalination, blue energy harvesting, or mineral extraction.

### BRIEF DESCRIPTION OF THE DRAWINGS

FIG. 1 is a schematic diagram illustrating a platform for OIF using electrochemical processes in accordance with one or more embodiments.

FIG. 2 is a schematic diagram illustrating a platform for OIF with various alternate powering systems including tidal turbine, PV cells, and hydrogen fuel systems in accordance with one or more embodiments.

FIGS. 3A-3C are schematic diagrams illustrating a platform for OIF in different installation options in accordance with one or more embodiments: self-operating and drifted by the ocean waves (FIG. 3A), self-operating and tugged by a ship or ROV (FIG. 3B), and carried out by a ship (FIG. 3C).

FIGS. 4A-4C are schematic diagrams illustrating a platform for OIF with different electrode configurations in accordance with one or more embodiments: underwater anode/cathode with vertical facing orientation (FIG. 4A), floating cathode/underwater anode with horizontal stacking orientation (FIG. 4B), and floating anode/cathode with horizontal facing orientation (FIG. 4C).

FIGS. 5A-5D are schematic diagrams illustrating a platform for OIF with different electrode shapes in accordance with one or more embodiments: flat plates (FIG. 5A), rods (FIG. 5B), porous plates (FIG. 5C), and net cathode with spherical/disk anode (FIG. 5D).

### DETAILED DESCRIPTION

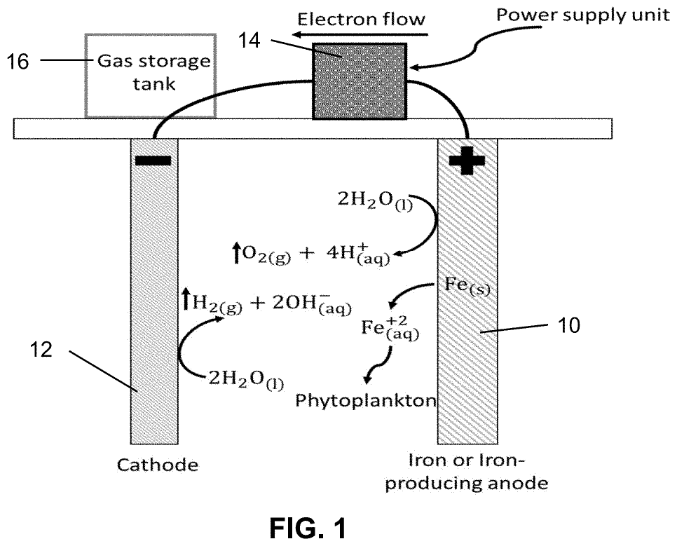

FIG. 1 illustrates an exemplary offshore mobile platform for electrochemical OIF and H₂ production in accordance with one or more embodiments. The generated iron via the electrochemical reactions is directly dissolved into the ocean in its ferrous form to become available to the phytoplankton. Unlike previous OIF techniques, the electrochemical approach in accordance with one or more embodiments can produce bioavailable iron without preprocessing (e.g., making iron powder1) or any additional chemical reactions that can impact the ocean environment. The system includes four main components: anode 10, cathode 12, power supply unit 14, and gas storage tank 16. The chemical reactions occur at the electrodes 10, 12 to produce iron and H₂ gas as follows:

At the anode 10, iron electrolysis takes place using either an iron or iron-production electrode. The standard potential of this electrolysis reaction is 0.44 V2. The reaction is given as:

Fe₍ₛ₎→Fe_{(aq)}⁺²+2e⁻.

In addition, O₂ evolution reaction can occur at the anode as follows:

2H₂O₍ₗ₎→O_{2(g)}+4H_{(aq)}⁺+4e⁻.

The generated oxygen gas can be collected and stored. Or, it can leave the ocean surface to the atmosphere.

At the cathode 12, seawater splitting takes place to produce H₂ gas. The produced gas is be collected and stored using the installed tank at the platform as shown in FIG. 1. The water-splitting reaction is given as:

2H₂O+2e⁻→H_{2(g)}+2OH_{(aq)}⁻.

The formed OH— increases the water's alkalinity. The overall reactions at the anode 10 and cathode 12 can involve additional secondary reactions as will be discussed below.

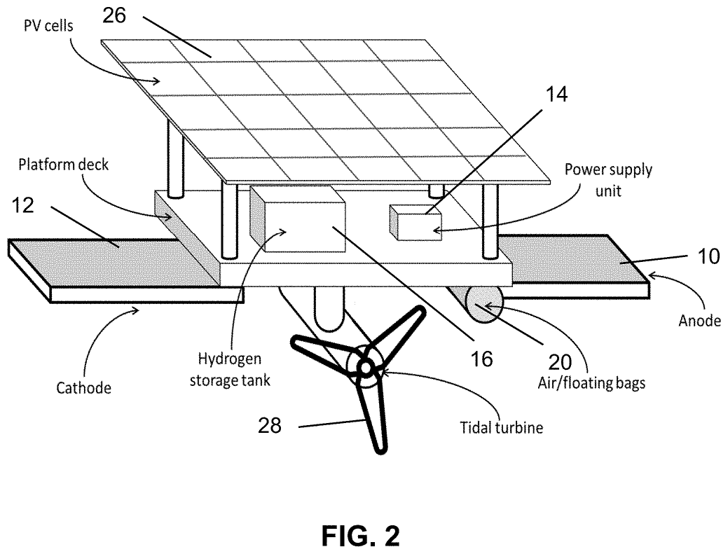

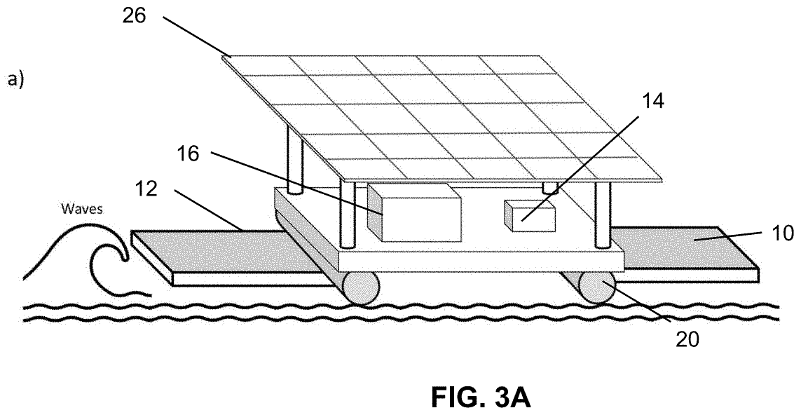

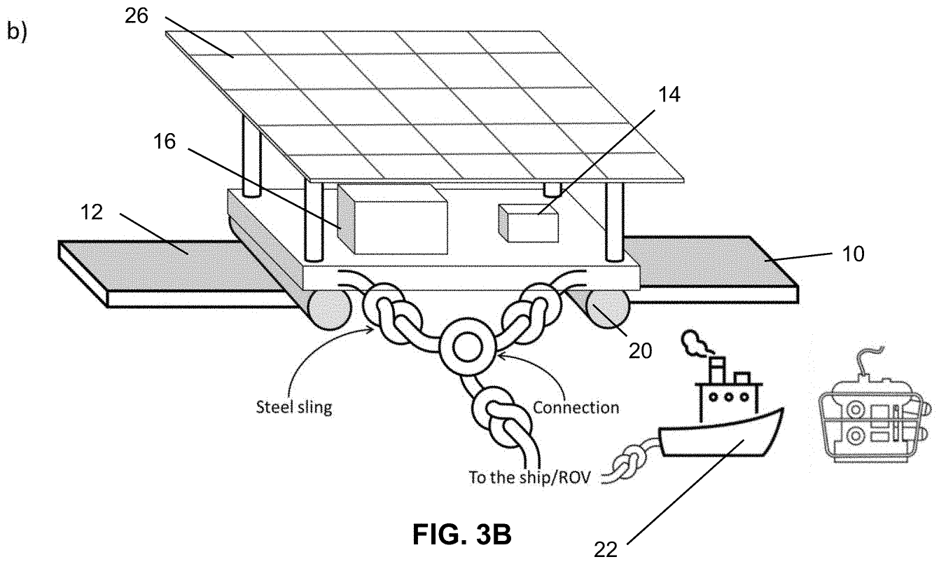

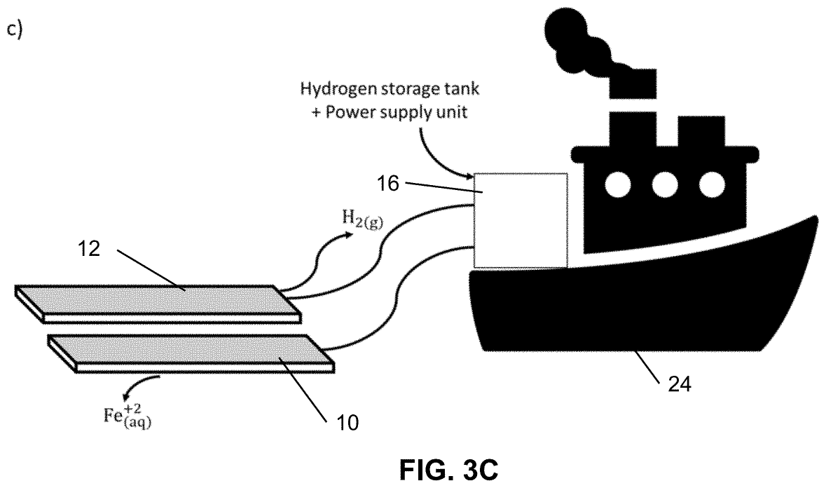

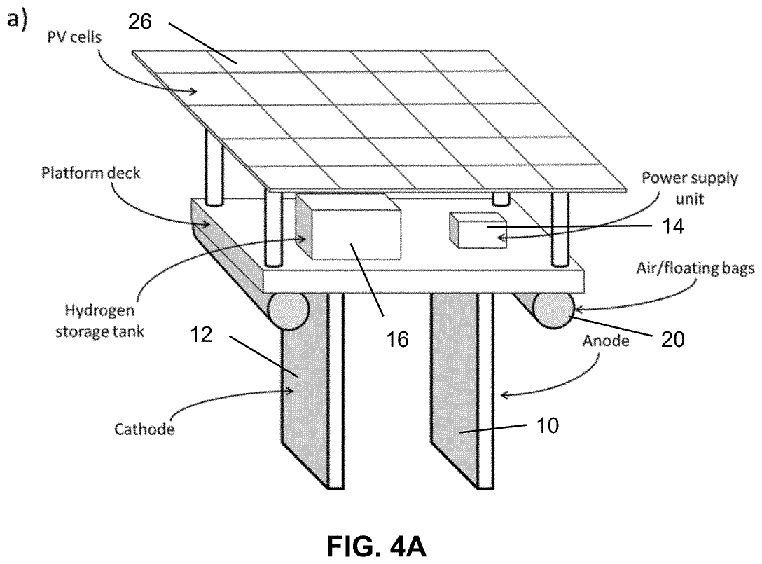

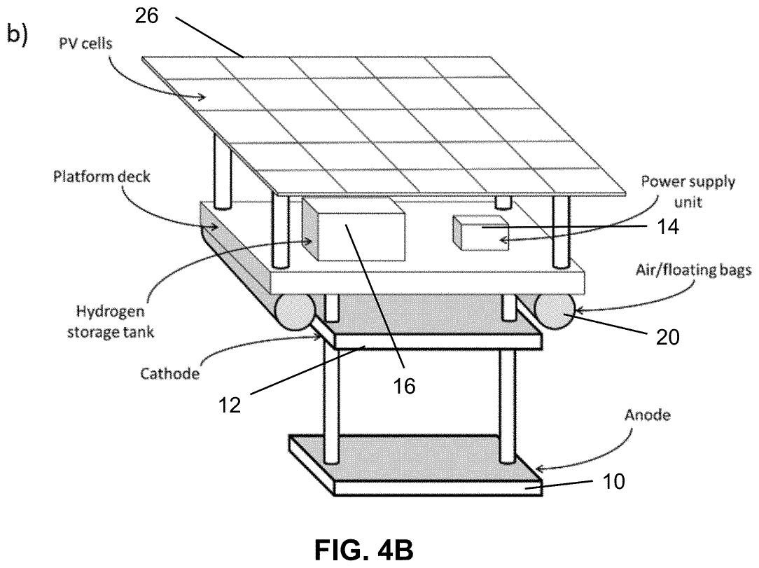

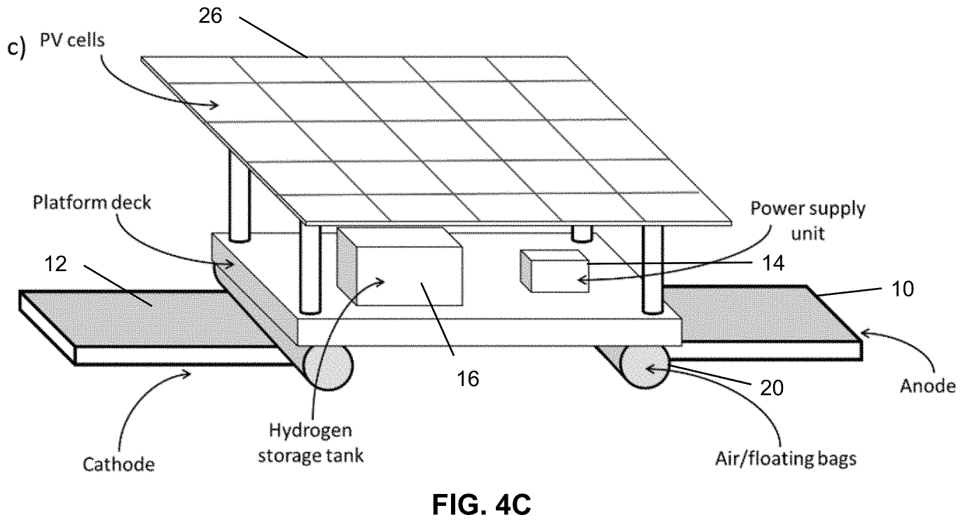

These electrochemical reactions are electrically powered by either a traditional electrical generator, a renewable energy device (e.g., solar PV cells 26, a tidal turbine 28, blue energy harvesting), or by reusing the generated hydrogen as fuel. FIG. 2 illustrates some of these options. The platform design promotes sustainability and environmentally-friendly techniques to eliminate any harmful impact on the ocean or atmospheric environment. In addition, the platform can be designed with high flexibility and dynamicity. The platform structure can be either floating in the ocean or carried out on a ship/barge as shown in FIGS. 3A-3C. In the FIG. 3A embodiment, the platform is self-operating and floating using an installed device (e.g., marine salvage or pillow lift bags 20). The load of the lift bags 20 will be equal to or more than the weight of the platform to keep it floating. In this case, the top part of the platform (e.g., deck) is above the ocean surface and the electrodes 10, 12 can be either floating or underwater as shown in FIGS. 4A-4C. The power supply unit 14 and gas storage tank 16 are installed on the platform deck. The floating platform can be either driven by the ocean currents (FIG. 3A) or tugged by a boat or ROV 22 (FIG. 3B) to distribute the generated iron in the ocean. Alternatively, the platform can be carried out by a ship 24 and the electrode parts as illustrated in FIG. 3C. In this case, the platform deck will be operated from the ship 24.

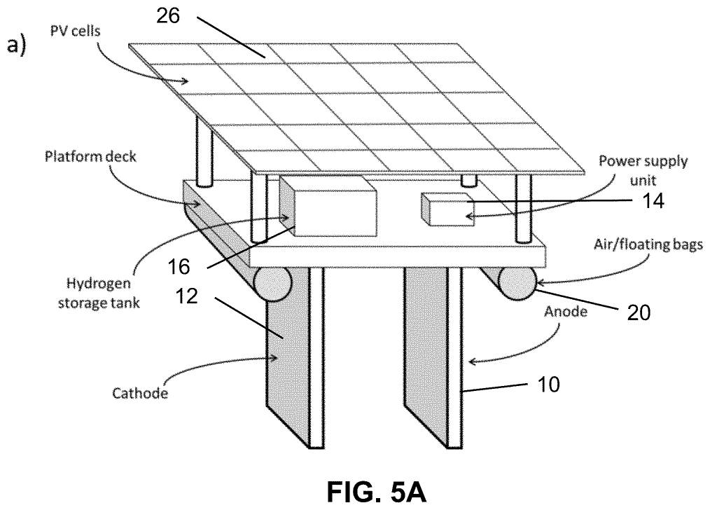

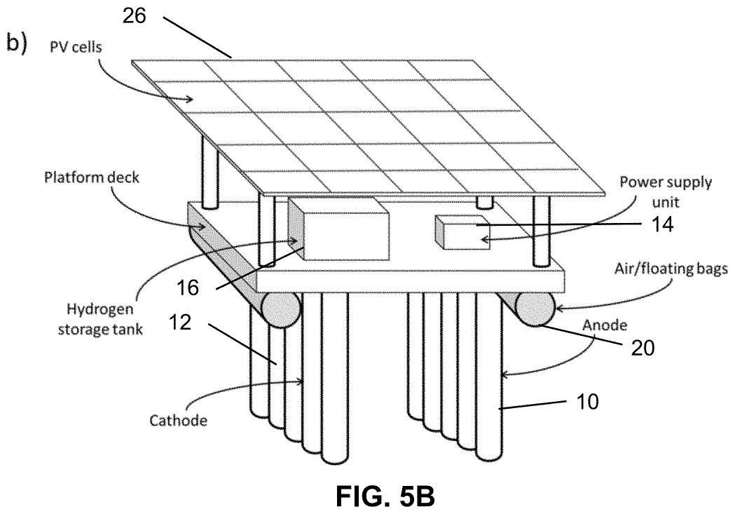

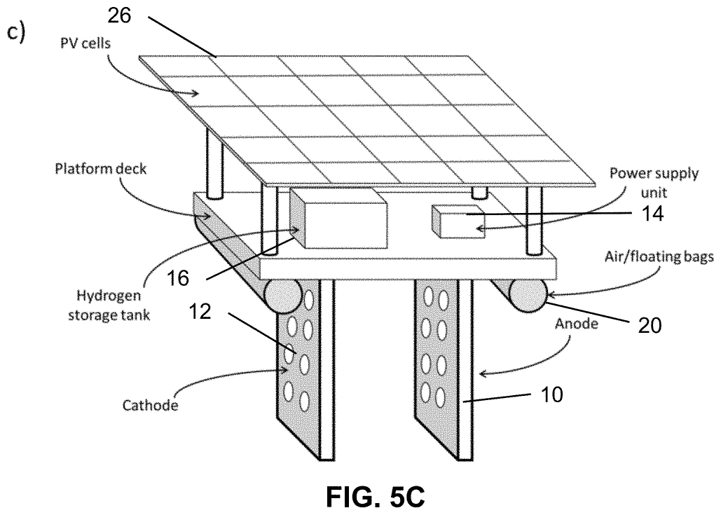

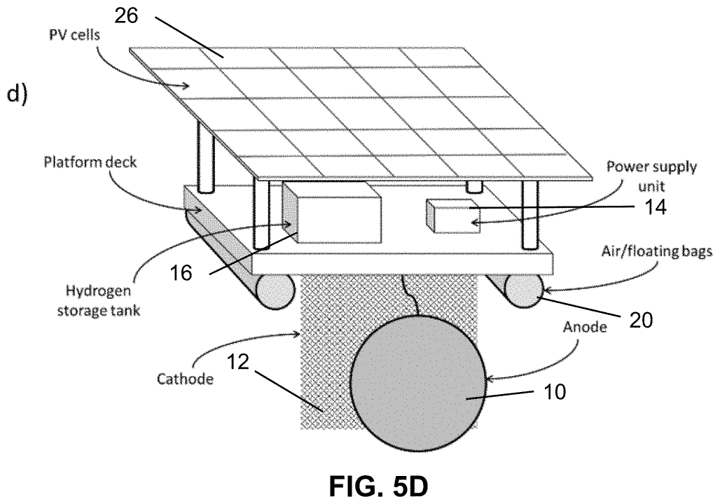

The electrodes 10, 12 can have various configurations and shapes to optimize the electrochemical processes and the engineering of the platform. The anode 10 and cathode 12 can be installed in various facing orientations such as vertical or horizontal configurations as exhibited in FIGS. 4A-4C. The optimized orientation can be determined by engineering several aspects including the direction of the electrochemical transport. Further, the anode 10 and cathode 12 can have many shape forms including cylindrical rods, nets, plates, perforated plates, disks, or spheres as shown in FIGS. 5A-5C. For instance, a net shape electrode can be suitable for floating electrode design, and an array of cylindrical rods electrode can be easier for offshore handling. Depending on the configuration and the shape of the electrode, the electrochemical processes can be applied in 1D, 2D, or 3D arrangements. In other words, the transport of the electrochemical species is determined by the dimensionality of the generated electric field which depends on the anode/cathode configuration and shape. The anode and cathode can have different shapes to generate a more complex electric field arrangement to tune the dimensionality of the electrochemical process.

For material selection, the anode 10 can be made from either iron or iron-producing materials. The cathode 12 can be made from iron, steel, or any other material. Both the anode 10 and cathode 12 can be made from iron with polarity reversal³. Additional materials, if necessary, can be used to enhance the chemical conditions such as using Aluminum. Finally, the electrolysis reaction and its mass transfer rate can be primarily controlled by manipulating the applied current at the electrodes. Continuous, intermittent, or time-varying current profiles can be applied. For example, a simple pulse current or temporal function of current application/reversal can be controlled using a controller associated with the power supply unit at the platform.

### Feasibility Analysis

We computed the needed iron and electricity to increase the ocean iron concentration within the experimentally reported values4. We aim to increase the ferrous iron concentration (Fe⁺²) by 1 nM over a surface area of 100×100 km and depth of 30 m. Therefore, we performed theoretical analysis as follows:
1) The volume will be V=100×100×10⁶×30=3×10¹¹ m³ or 3×10¹⁴ L.
2) The needed mass of iron will be m=1×10⁻⁹×3×10¹⁴=3×10⁵ mole or 16.75 ton.
3) Using Faraday's law, we can determine the needed current such as:

$$I = \frac{m \times n \times F}{t \times {M}_{w}} ,$$

where t is the reaction's time which is assumed to be five years (i.e., t=15.77×10⁷s), M_{w} is the molecular mass of iron, n is the number of participating electrons, and F is Faraday's constant. Therefore, the needed current is I=(2×96,485 C/mole)×3×10⁵ mole/(15.77×10⁷s)=367 A.

We can install a 20 kW PV system of series panels to obtain the needed current. By assuming that each panel can output a current of ˜6-8 A, we roughly need about 70 panels to power the platform. The 70 panels will have a total equivalent area of 1400 sq ft with a cost of ˜$55,000. These calculations show the feasibility of our practical invention. We should highlight that the needed 16.75 tons can be supplied by running several platforms at the same time. This will reduce the overall needed time to complete the OIF process.

### Secondary Reactions

The main electrochemical reactions in the platform are shown in FIG. 1. However, secondary reactions can occur depending on the pH, presence of various ions, and other environmental conditions. These reactions will mainly affect the bioavailability of the generated ferrous irons for the phytoplankton. For example, ferric iron (i.e., Fe_{(aq)}⁺³)) or iron oxide (i.e., Fe(OH)₂₍ₛ₎) can form by hydrolysis or oxidation processes of Fe_{(aq)}⁺² depending on the pH level^{2,5}. In addition, chloride ions can react at the anode to form chlorine gas or are adsorbed at the anode by the surface polarization6,7. At the cathode, secondary reactions can also occur such as converting the formed OH⁻ to O₂ gas. Therefore, the environment of the reactions should be controlled to reduce unnecessary secondary reactions. Our invented platform provides high flexibility to control the reaction conditions.

Several approaches can be implemented to enhance the bioavailability of the produced iron at the anode 10. First, the electrode materials can be optimized to control the electron transfer and surface polarization conditions. For example, layered7 or hybrid electrodes can be used to enhance the chemical conditions if necessary. An array of alternating rods (e.g., Fe—Al—Fe—Al- . . . etc) can be used to improve the reaction conditions as shown in FIG. 5B. Further, adding catalysts or selective membranes near the anode can reduce the reach of specific species to the anode (e.g., chloride ions). The pH level of the reaction medium can also be controlled by optimizing the production rate of OH⁻ at the cathode (e.g., applying the current as a function of time). Finally, electrochemical electrolysis can be performed under forced convection conditions. In other words, the driving speed of the platform by an external ship/ROV will impose a high convection rate on the electrochemical transport. The convection direction can be either parallel or perpendicular to the direction of the electrodes depending on the stacking configuration (see FIG. 4). Such forced convection conditions can ensure a fast mass transport rate and mixing of the produced iron at the anode before the occurrence of any additional hydrolysis or oxidation. Also, it can help to avoid increasing the pH level beyond the desirable value at the anode by driving the produced OH⁻ at the electrode away from the anode.

Understanding the bioavailability and the reaction conditions of OIF and H₂ gas production in seawater is crucial to engineer and optimize the efficiency of the platform. Computational tools can be used to investigate the fundamental aspects of these conditions. We can use computational chemistry simulations to study the molecular and atomistic features of the interacting chemical species. Also, computational fluid dynamics can be coupled with the ecosystem characteristics to model the chemical rates and transport of the produced chemical species in the ocean. The coupled ecosystem models can involve the growth and CO₂ capturing by the phytoplankton system.

Having thus described several illustrative embodiments, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions. Accordingly, the foregoing description and attached drawings are by way of example only, and are not intended to be limiting.

### REFERENCES

1 Emerson, D. Biogenic Iron Dust: A Novel Approach to Ocean Iron Fertilization as a Means of Large Scale Removal of Carbon Dioxide From the Atmosphere. Frontiers in Marine Science 6, doi:10.3389/fmars.2019.00022 (2019).
2 Lakshmanan, D., Clifford, D. A. & Samanta, G. Ferrous and ferric ion generation during iron electrocoagulation. Environ Sci Technol 43, 3853-3859, doi:10.1021/es8036669 (2009).
3 Mao, X., Baek, K. & Alshawabkeh, A. N. Iron Electrocoagulation with Enhanced Cathodic Reduction for the Removal of Aqueous Contaminant Mixtures. Environ Eng Manag J 14, 2905-2911 (2015).
4 Sunda, W. G. & Huntsman, S. A. Interrelated influence of iron, light and cell size on marine phytoplankton growth. Nature 390, 389-392, doi:10.1038/37093 (1997).
Matheson, L. J. & Tratnyek, P. G. Reductive dehalogenation of chlorinated methanes by iron metal. Environ Sci Technol 28, 2045-2053, doi:10.1021/es00061a012 (1994).
6 Huang, C.-H. et al. Effect of Chloride Ions on Electro-Coagulation to Treat Industrial Wastewater Containing Cu and Ni. Sustainability 12, doi:10.3390/su12187693 (2020).
7 Kuang, Y. et al. Solar-driven, highly sustained splitting of seawater into hydrogen and oxygen fuels. Proc Natl Acad Sci USA 116, 6624-6629, doi:10.1073/pnas.1900556116 (2019).

1. An ocean iron fertilization (OIF) system for electrochemically controlled release of iron in an ocean to stimulate growth of phytoplankton to increase CO₂ sequestration by the ocean, comprising:
a cathode submerged or floating in the ocean;
an iron or iron-producing anode submerged or floating in the ocean spaced apart from the cathode;
a power supply unit connected to the cathode and the anode for driving electric current between the cathode and the anode such the anode generates oxygen (O₂) and ferrous iron through electrolysis to be released in the ocean, and the cathode produces hydrogen (H₂) and hydroxide (OH—) species through an electrochemical reaction at the cathode.

2. The system of claim 1, further comprising a collector for collecting the H₂, and a gas storage unit for storing the H₂.

3. The system of claim 1, wherein the OH— is released to the ocean to enhance alkalinity of the ocean via increasing the pH level of the ocean.

4. The system of claim 3, wherein the increased pH level promotes de-acidifying the ocean and converting the CO₂ in the ocean to bicarbonate or carbonate irons, thereby increasing the ocean capacity to removal more atmospheric CO₂.

5. The system of claim 1, wherein the power supply unit comprises a mechanical power generator, a battery, or a renewable energy device.

6. The system of claim 5, wherein the renewable energy device comprises photovoltaic cells, a tidal turbine, or a blue energy device.

7. The system of claim 5, wherein the mechanical power generator is fueled by H₂ collected at the cathode.

8. The system of claim 1, wherein the system is configured for operation in a free-standing platform, a towable platform, or a boat.

9. The system of claim 1, further comprising a control system configured to operate the system continuously, intermittently, or as a function of time.

10. The system of claim 1, further comprising a control system configured to vary the magnitude of the electric current supplied by the power supply unit to control iron flux release.

11. The system of claim 1, wherein the cathode and the anode operate in a vertical or a horizontal orientation.

12. The system of claim 1, wherein the cathode and the anode comprise cylindrical rods, nets, plates, perforated plates, disks, or spheres.

13. The system of claim 1, wherein the cathode comprises aluminum, iron, or steel.

14. The system of claim 1, wherein the cathode and the anode are layered.

15. The system of claim 1, further comprising catalysts or selective membranes proximate to the anode to reduce the reach of given species to the anode.

16. An ocean iron fertilization (OIF) method for electrochemically controlled release of iron in an ocean to stimulate growth of phytoplankton to increase CO₂ sequestration by the ocean, comprising:
submerging or floating a cathode in the ocean;
submerging or floating an iron or iron-producing anode in the ocean spaced apart from the cathode;
driving electric current between the cathode and the anode using a power supply such the anode generates oxygen (O₂) and ferrous iron through electrolysis to be released in the ocean, and the cathode produces hydrogen (H₂) and hydroxide (OH—) species through an electrochemical reaction at the cathode.

17. The method of claim 16, further comprising varying the magnitude of the electric current to control iron flux release.

18. The method of claim 16, further comprising causing selective iron release reactions to change the form of the released iron for increasing iron bioavailability.

19. The method of claim 16, further comprising collecting the H₂ in a gas storage unit.

20. The method of claim 16, further comprising varying the magnitude of the electric current supplied by the power supply unit to control iron flux release.
